(19) United States
(12) Patent Application Publication
(10) Pub. No.: US 2019/0352692 A1
(43) Pub. Date: Nov. 21, 2019

(54) SUBSTRATE IMPREGNATED WITH AT LEAST ONE COMPOSITION FOR THE SAMPLING OF MICROORGANISMS PRESENT ON A SURFACE

(71) Applicant: REALCO, Louvain-la-Neuve (BE)

(72) Inventors: Gordon Blackman, Lasnes (BE); Sébastien Fastrez, Mons (BE); Camille Gathy, Ceroux-Mousty (BE)

(73) Assignee: REALCO, Louvain-la-Neuve (BE)

(21) Appl. No.: 16/314,354

(22) PCT Filed: Jun. 27, 2017
(86) PCT No.: PCT/EP2017/065792

§ 371 Date: Dec. 28, 2018

(30) Foreign Application Priority Data
Jun. 29, 2016 (BE) 2016/5499

(51) Int. Cl. C12Q 1/06; C12Q 1/02; C12Q 1/18; C11D 17/00; C11D 3/04; C11D 3/22; C11D 3/37; C11D 3/386; C11D 3/48

(52) U.S. Cl. CPC C12Q 1/06; C12Q 1/025; C12Q 1/18; C11D 17/003; C11D 3/48; C11D 3/222; C11D 3/3765; C11D 3/386; C11D 3/046

(57) ABSTRACT

The invention relates to a substrate impregnated with at least one composition comprising at least one enzymatic component for the sampling of microorganisms present on a surface, in particular for the sampling of microorganisms that may or may not have formed a biofilm on said surface, said at least one composition having a dynamic viscosity of between 50 and 2000 mPa·s.

The present invention relates to a substrate impregnated with at least one composition comprising at least one enzymatic component for the sampling of microorganisms present on a surface, in particular for the sampling of microorganisms having formed or not formed a biofilm on said surface.

The term “impregnated substrate” refers to, according to the present invention, a substrate in which a liquid spreads out or diffuses in all parts. The term saturated substrate may also be used, the terms “impregnated” and “saturated” being used interchangeably according to the present invention.

In numerous fields of work, such as the agri-food sector, rural communities, medical and veterinary sectors, problematical contaminations due to the presence of microorganisms are very often observed. Yet, in these days, strict hygiene standards are required and it must be ensured that the number of microorganisms responsible for such contaminations be kept below an acceptable threshold value, this threshold value being specific for each field of work. Typically, in hospitals and medical and veterinary practices, the presence of microorganisms is responsible for nosocomial infections while, in agri-food and rural communities, these microorganisms are responsible for the deterioration of perishable goods but also the transfer of contaminants to the consumers of the products coming, for example, from a meat production chain.

Planktonic bacteria, that is the bacteria free on a liquid or solid substrate, in themselves pose a problem as they are able to directly contaminate any type of surfaces such as food, medical tools or human or animal patients. However, it is largely recognised today that the problems of contamination by these microorganisms are particularly significant as the latter form biofilms.

A biofilm is a slime layer which grows on all surfaces, following the adhesion of microorganisms on these surfaces and the secretion therefrom of polymers which coat them and facilitate their adhesion. The biofilms thus form a layer of protection around the microorganisms and represent a recurrent source of contamination of the surrounding environment which poses problems, for example in the food industry and in hospital environments.

More specifically, the accumulation of polymers secreted by the bacteria creates a matrix essentially formed of polysaccharides, DNA, proteins as well as lipids, which protects these microorganisms from external aggressions and is very resistant to conventional cleaning and disinfecting procedures. The microorganisms therefore develop easily within this protective matrix and contaminate the surrounding environment by forming a particularly critical reservoir which is difficult to eliminate.

It is known that the problem of the presence of biofilms is twofold. Firstly, as indicated above, they represent a permanent source of contamination which is very difficult to eliminate by conventional means, even by the most aggressive means. Indeed, common disinfectants are often ineffective as it has been observed that they do not reach the microorganisms which are protected by the biofilm matrix formed of polysaccharides, DNA, proteins and lipids.

Secondly, a biofilm is generally mixed, in that it is initially developed by certain bacterial strains, but it may accommodate others, these strains living and developing in colonies. However, these colonies promote communication between bacteria and, among other things, the exchange and spreading of resistance genes carried by certain bacteria. The biofilms formed by these gene exchanges are thus more difficult to eliminate and increasingly powerful means of disinfection or treatment must be resorted to, which, however, frequently face major problems of resistance and/or tolerance.

In hospital and veterinary environments, but also in agri-food environments, the situation is all the more critical as numerous microorganisms responsible for the formation of biofilms are detected in numerous places. In hospital and veterinary practices, biofilms are detected around individuals (patients and animals) as well as in the surrounding area (operating room, surgical tools, equipment for maintaining said tools, endoscopes, urinary tubes, catheters, medical equipment, dialysis or assisted breathing machines for individuals, etc.) and on surfaces (floors, walls, operating tables, etc.). In the agri-food field, and particularly in factories for manufacturing food products, biofilms can be found around machines, tables, packaging and on operators even if they take all precautions possible in order to avoid contaminating the surfaces and work tools.

From all of this, it appears that biofilms constitute a real problem, particularly in the fields of healthcare (hospitals, dental practices, etc.), veterinary care and agri-food. This problem is all the more critical as biofilms may involve bacteria responsible for infections which could be fatal in individuals, if these bacteria are present in hospitals, veterinary practices or in food products.

As indicated above, the problem of the biofilm is twofold. On the one hand, common disinfectants are very often ineffective because it has been observed that they are not able to reach the microorganisms which are protected by the matrix of the biofilm formed of polysaccharides, DNA, proteins and lipids. On the other hand, a biofilm being generally mixed, it may contain several bacterial strains thus promoting the spreading of resistance genes between the different strains present within the biofilm and thus rendering their treatment very difficult or ineffective.

Consequently, from one environment to another or from one field of work to another, it is not rare and actually quite frequent that the biofilms detected are totally different. In order to detect the biofilms, kits for detecting the presence of biofilms on surfaces or in more specific installations (water systems, etc.) exist, these kits (such as that disclosed in document EP2537601) allowing staining of the biofilms to be carried out. It is thus currently possible to determine the zones in which biofilms are present in order to carry out their removal without, however, knowing the precise nature of the microorganisms (bacteria) at the origin of the targeted biofilm.

As a result, compositions for removing biofilms comprising multiple enzymes are used without, however, knowing if the enzymes used and optionally formulated in a detergent base (see, for example, document EP2243821) are really appropriate to act on a given biofilm. For this reason, the current treatments are more random and non-specific, which is not economically viable.

Furthermore, techniques for sampling microorganisms from surfaces have been developed in order to characterise and/or quantify the microorganisms present on the surfaces and responsible for contaminations. These sampling methods thus allow, on a surface, different types (of strains) of bacteria and microorganisms present to be determined.

Among the methods for sampling microorganisms present on a surface, one of the references on the subject is the ISO standard 18593:2004(F) which provides and defines horizontal methods for techniques for sampling from surfaces (more specifically from surfaces found in the environment of the agri-food industries). The “cloth/sponge” method is an example of sampling techniques cited in this standard, this method allowing the microorganisms present on a surface to be detected and quantified. Briefly, according to this sampling method, this involves dampening the cloth/sponge with a quantity of diluent which is from a physiologically sterile serum, sampling the surface in two perpendicular directions before placing the cloth/sponge into a sterile recipient with the diluent. Subsequently, following a potential storage of the sample, it is quantitatively and/or qualitatively analysed.

Unfortunately, if such a method allows sampling of microorganisms free on the surface of a substrate to be ensured, that is sampling of planktonic bacteria, it turns out to be ineffective when the surface is contaminated by microorganisms having formed a biofilm thereupon. In fact, as explained above, the matrix of the biofilm formed of polysaccharides, DNA, proteins and lipids forms a polymeric layer on the microorganisms, this matrix forming a real barrier protecting the microorganisms against external agents (chemical and/or physical).

As mentioned above, certain current techniques for sampling microorganisms depend on the use of substrates impregnated with water, as is the case for the substrates described in ISO standard 18593:2004(F). However, the simple fact of sampling a surface in two perpendicular directions using, for example, a cloth/sponge impregnated with physiologically sterile water, does not allow a sampling representative of all the microorganisms contaminating a surface to be ensured.

Furthermore, it has been found that the sampling techniques depending on a substrate impregnated with water may, in contrast, have a beneficial effect on the development of the microorganisms even following the undertaking of a sampling from a surface. In fact, it is known that certain bacteria exhibit increased growth in the presence of water, the substrate saturated with water thus promoting this growth during the sampling but also after the sampling as a thin film of water remains on the treated substrate where the sampling took place. This may thus promote the development of certain microorganisms not collected by the cloth/sponge.

Document WO98/37229 relates to a method and a device for sampling microorganisms from a surface in order to verify their properties, the device comprising a substrate impregnated with an enzyme, i.e. luciferase. More particularly, it is a rod which has an absorbent material at its ends, in which luciferase and luciferin are fixed. By moving the rod over a surface, a sampling of the microorganisms present there is taken and, subsequently, the ATP is detected according to the luciferin/luciferase bioluminescence reaction, which allows the cleanliness of the surface in terms of the presence of microorganisms to be determined.

Unfortunately, with such a sampling device, it appears that the microorganisms having formed a biofilm are not effectively collected. As a result, the sampling of the microorganisms from a surface is not representative of the bacterial populations which are nonetheless present there.

The object of the invention is to overcome the disadvantages of the prior art by obtaining a substrate allowing all the microorganisms present on a surface to be effectively, in an appropriate and representative manner, collected. In particular, the object of the invention is to obtain a substrate allowing not only the free (planktonic) microorganisms but also the microorganisms having formed a biofilm on said surface to be collected, without the sampling method using such a substrate according to the invention being more restrictive or necessarily longer (length of application of the substrate to the surface where the sampling is carried out).

The invention thus aims to obtain a device and a method allowing a simultaneous sampling of the free (planktonic) bacteria and the microorganisms having formed a biofilm on said surface to be carried out, so the sampling is representative of the populations of microorganisms contaminating a surface, this certainly not being the case when sampling with a substrate impregnated with physiological water or during a sampling carried out with a device according to document WO98/37229.

To solve this problem, a substrate is provided according to the invention, impregnated as indicated at the beginning, characterised in that said at least one composition has a dynamic viscosity of between 50 and 2,000 mPa·s.

Advantageously, said at least one composition has a dynamic viscosity of between 100 and 1,000 mPa·s, preferentially between 200 and 800 mPa·s, more preferentially between 300 and 600 mPa·s, still more preferentially between 400 and 500 mPa·s.

In the scope of the present invention, it has been determined that substrate impregnated with a composition comprising at least one enzymatic component and which has a dynamic viscosity of between 50 and 2,000 mPa·s allows a significantly greater number of microorganisms, that is both the free microorganisms (planktonic bacteria) and the microorganisms (bacteria) responsible for the formation of biofilms protecting them, to be collected/sampled. In contrast to the common sampling techniques and methods as described above, a substrate according to the invention allows a genuinely representative sampling of the microorganisms present on a surface to be taken and not only a sampling of the free (planktonic) bacteria.

Surprisingly, it has been determined that impregnating/saturating a fabric-type substrate (or any other type of substrate) with an enzymatic composition according to the invention allows bacteria and other microorganisms whose presence on a surface was neither expected nor suspected to be collected. In fact, with a substrate according to the invention, it has been shown:
that free microorganisms are collected in a way at least equivalent with respect to those collected with the sampling techniques of the prior art (impregnated sampling substrate), and
that microorganisms not collected by the commonly used substrates are collected with a substrate according to the invention.

As a result, the use of a substrate according to the invention, in contrast to current substrates, allows a genuinely representative and complete sampling of the contaminants present on a surface to be obtained. Consequently, the use of a substrate according to the invention allows, subsequently, a treatment for removing biofilms which is targeted and effective to be able to be applied, by using the appropriate compounds and no longer a cocktail of compounds of which some are not useful against a given contamination. This has a considerable economic advantage.

The appeal of a substrate according to the invention is thus doubled in the sense that it allows all the contaminants on a surface to be sampled and it then allows precisely which treatment for removing the microorganisms to apply to be determined. Finally, with a substrate according to the invention, an appropriate and targeted removal of all the microorganisms (planktonic and protected by a biofilm) may be undertaken downstream of a sampling with this substrate impregnated with an enzymatic composition according to the invention.

According to the invention, the sampling substrate thus allows the microorganisms whose presence was not detected with the common sampling substrates and methods, such as the “cloth/sponge” method where the substrate is saturated with physiological water or the method according to document WO98/37229, to be highlighted, that is collected and identified. Consequently, the invention truly allows the sampling of microorganisms from a surface to be improved, by means of a substrate impregnated with a composition comprising at least one enzymatic component and having a dynamic viscosity of between 50 and 2,000 mPa·s, the sampling carried out not being more restrictive nor necessarily longer than a common sampling.

In particular, in the scope of the present invention, it has been highlighted that a viscous composition according to the invention, that is a composition comprising at least one enzymatic component and having a dynamic viscosity of between 50 and 2,000 mPa·s, allows microorganisms on surfaces arranged along varied axes to be collected. In fact, the viscous aspect of the impregnated substrate according to the invention allows the latter to “stick”/adhere to a surface and may, consequently, be placed on vertical surfaces or “upside down”, for example, under a workplan. The viscous aspect of the impregnated substrate according to the invention also allows the microorganisms to be “detached” and to “fix” them to the impregnated substrate in an optimal manner: the dynamic viscosity of a composition according to the invention allows the effectiveness of sampling microorganisms from a surface, both vertical and horizontal, to be improved and ensures that the sampling carried out is representative of the bacterial populations genuinely present on the surfaces forming the object of the sampling.

According to the invention, the viscous composition, such as a gel, may be obtained by mixing several surfactants, whether anionic, non-ionic or amphoteric, optionally with a mineral compound such as, for example, sodium chloride. The gel, that is the viscous composition, may also be obtained by the addition of compounds such as, for example, natural or synthetic polymers (xanthan derivatives, cellulose, polyacrylate, etc.).

Furthermore, it has been determined, in the scope of the present invention, that a viscous composition having a value of dynamic viscosity as indicated above allows the contact time necessary with the biofilms present on a surface to be significantly reduced in order to break them down sufficiently to release the bacteria and thus be able to collect and/or identify them. It has been shown that the composition being viscous genuinely allows it to stay in place and encompass the biofilms which are still present in the form of fine layers corresponding to “protrusions” in the order of several micrometres of thickness, this staying in place not being so optimal with a non-viscous composition. In other words, the dynamic viscosity of the composition comprising at least one enzymatic component in the scope of the present invention allows the biofilms to be entirely surrounded and to break them down more quickly and more effectively, the viscosity of the composition also contributing to “trapping” the microorganisms (bacteria) collected from a surface. Furthermore, with a viscous composition according to the invention, the problem of rapid evaporation of liquid compositions is not encountered.

Preferably, according to the invention, said at least one enzymatic component comprises at least one protease and/or at least one laccase and/or at least one polysaccharidase. In the scope of the present invention, it has been highlighted that use of such enzymes allows the biofilms to be effectively broken down.

Preferentially, said at least one enzymatic component comprises at least one additional enzyme chosen from the group formed of oxidoreductases, lyases (pectate lyase, etc.), transferases, peptidases (metalloprotease, serine-protease, exopeptidase, endo-protease, cystine-protease, etc.), lipases and esterases (lysophospholipase, phospholipase, etc.) and glucosaminidases.

Preferably, according to the invention, said impregnated substrate is a wipe or a sponge or a cloth or any other type of appropriate substrate. In the scope of the present invention, the substrates may be impregnated with at least one composition according to the invention and allow microorganisms to be attached to the substrate.

Advantageously, according to the invention, said at least one composition comprising at least one enzymatic component has a pH of between 5 and 11.

In a particularly advantageous embodiment of the device according to the invention, said substrate and said at least one composition comprising at least one enzymatic component are sterile. In fact, it is advantageous that said impregnated substrate along with said at least one composition comprising at least one enzymatic component are sterile so that other external contaminants do not distort the analysis of the collected microorganisms both from a qualitative and quantitative point of view, the objective being to carry out a sampling faithfully reflecting the contaminations of microorganisms on a surface.

Advantageously, according to the invention, said at least one composition comprising at least one enzymatic component further comprises at least one detergent component.

Preferably, according to the invention, said at least one detergent component comprises at least one wetting agent and/or at least one sequestering agent and/or at least one dispersing agent.

The wetting agent of the detergent component is an amphiphilic chemical substance, or a composition comprising said amphiphilic chemical substance, which modifies the surface tension between two surfaces. The wetting agent has an advantage of promoting the spreading of a liquid over a solid but also of furthering the contact between two surfaces. More particularly, the wetting agent furthers contact between the detergent component and a surface and, consequently, between the enzymes and their substrate. For example, on the stainless-steel surfaces frequently found in the agri-food industries but also in hospital or veterinary environments, the wetting agent allows a homogeneous spreading of the composition to be carried out and thus its perfect distribution over the surfaces to be decontaminated, for example, on the production tools, workplans, floors or operating tables and medical tools.

The wetting agent may be anionic, cationic, non-ionic or zwitterionic. Preferentially, the wetting agent may be an anionic or non-ionic wetting agent, that is the hydrophilic part is negatively charged or bears no net charge, or may be a composition comprising an anionic wetting agent. More particularly, the wetting agent may be a sucrose ester or a composition comprising a sodium alkyl sulphate and an alcohol.

The sequestering agent is a chemical substance having the capacity to form complexes with mineral ions which it arranges in a form preventing their precipitation by standard reactions. For example, the sequestering agent may be ethylenediaminetetraacetic acid, glucono delta-lactone, sodium gluconate, potassium gluconate, calcium gluconate, citric acid, phosphoric acid, tartaric acid, sodium acetate, sorbitol, a compound comprising a phosphorous atom. Preferentially, the sequestering agent may be a phosphorous oxide such as a phosphonate, a phosphinate or a phosphate or mixtures thereof, or a salt thereof, an amine or an amine oxide having at least, in its structure, a functional phosphine group, phosphine oxide, phophinite, phosphonite, phosphite, phosphonate, phosphinate or phosphate, alone or in combination, or a salt thereof.

More preferentially, the sequestering agent may be a phosphonate or a salt there of, an amine or an amine oxide comprising at least, in its structure, a functional phosphine group, phosphine oxide, phosphinite, phosphonite, phosphite, phosphonate, phosphinate or phosphate, alone or in combination, or a salt thereof. As a non-limiting example, the phosphonate may be of general formula R¹(R²O)(R³O)P═O, wherein R¹, R² and R³ independently represent a hydrogen, alkyl, substituted alkyl, substituted alkyl-amino or not, substituted aminoalkyl or not, aryl or substituted aryl group. As a non-limiting example, the amine or amine oxide may comprise one, two or three substitute(s) of general formula CR⁴R⁵W, wherein R⁴ and R⁵ represent, independently from one another, a hydrogen, alkyl, substituted alkyl, substituted or non-substituted alkyl-amino, substituted or non-substituted aminoalkyl, aryl or aryl substituted group, and W represents a phosphonate, phosphinate or phosphate group.

The sequestering agent may be in the form of a sodium, calcium, lithium, magnesium or potassium salt; preferentially, the sequestering agent may be in the form of a sodium, calcium or potassium salt.

Preferably, the sequestering agent is an agent which may be used safely in the food industry, in that the sequestering agent is no risk to health, alone or combined with other components.

The dispersing agent of the detergent component allows the separation of the particles of a suspension to be improved in order to prevent agglutination, aggregation and/or decantation. This dispersing agent may be a polymer which is soluble or partially soluble in water such as, for example, polyethylene glycol, celluloses derivatives or a polymer comprising at least one acrylic acid or acrylic ester or polyphosphate unit. Preferentially, the dispersing agent is a polymer comprising at least one acrylic acid or acrylic ester unit of general formula —(CH₂—CH—COOR)—, wherein R represents an alkyl or substituted alkyl, aryl or substituted aryl, or hydrogen group. For example, the dispersing agent is a polymer having an average molecular weight Mw of approximately between 500 and 10,000.

More preferentially, the dispersing agent is an acrylic acid polymer. For example, the dispersing agent may be an acrylic acid homopolymer having an average molecular weight of approximately between 2,000 and 6,000.

The presence of a dispersing agent in the composition according to the invention thus allows any aggregation of bacterial particles during cleaning of surfaces to be avoided, which ensures an optimal removal of the particles of biofilms detached from a substrate under the action of the enzymes. In fact, rather than joining together, these particles remain separated in a suspension, not re-depositing or re-adhering to the cleaned surface.

Preferably, according to the invention, said at least one detergent component comprises a proportion of sequestering agent ranging between 1 and 10 wt %, a proportion of dispersing agent ranging between 1 and 10 wt % and a proportion of wetting agent ranging between 1 and 30 wt % with respect to the total weight of the detergent component. Preferably, said at least one detergent component comprises a proportion of wetting agent ranging between 5 and 20 wt %, preferentially a proportion of wetting agent equal to 15 wt % with respect to the total weight of the detergent component.

Other embodiments of a substrate impregnated with at least one composition are indicated in the appended claims.

The invention also relates to a method for sampling microorganisms present on a surface, in particular for sampling microorganisms having formed or not formed a biofilm on said surface, using a substrate according to the invention, said method comprising the following steps:
impregnating a substrate with a predetermined quantity of at least one composition comprising at least one enzymatic component and having a dynamic viscosity of between 50 and 2,000 mPa·s;
applying said impregnated substrate to said surface over a predetermined period of time in order to collect, from said surface, microorganisms having formed or not formed a biofilm thereupon; and
removing said substrate from said surface.

The impregnation of the substrate according to the invention may occur before application of the substrate to a surface or following application of the not yet impregnated substrate to a surface.

Advantageously, according to the invention, said predetermined period of time for application of said impregnated substrate to said surface ranges between 1 and 45 minutes and is preferably 15 minutes.

In a particular embodiment, said method according to the invention comprises a prior step of heating said at least one composition comprising said at least one enzymatic component to a temperature ranging between 15 and 65° C., preferably to a temperature of 45° C. This step of heating the enzyme allows the latter to be activated and achieve 100% of enzymatic activity.

Preferably, according to the invention, said method comprises an additional step of swabbing said surface, so the microorganisms not collected by said impregnated substrate are collected by means of swabs. Such additional sampling enforces the fact that the sampling carried out truly reflects the contaminations by microorganisms on a surface or a substrate. Alternatively, rather than carrying out swabbing, a substrate substantially identical to the impregnated substrate according to the invention but not impregnated may be used.

Advantageously, according to the invention, said method comprises an additional step of removing said collected microorganisms in a sterile removal solution.

In a particularly advantageous embodiment of the method according to the invention, said method comprises an additional step of quantifying and/or identifying said removed microorganisms.

Other embodiments of the method for sampling microorganisms present on a surface according to the invention are indicated in the appended claims.

The invention also relates to a kit for the sampling of microorganisms present on a surface, in particular for the sampling of microorganisms having formed or not formed a biofilm on said surface, said kit comprising:
a substrate, for example, a fabric-type substrate; and
a sample of at least one enzymatic component in solution or in solid form.

The sample of at least one enzymatic component has, in solution, a dynamic viscosity of between 50 and 2,000 mPa·s. When the sample of at least one enzymatic component is initially in solid form, dissolution of the latter is carried out to obtain an enzymatic component having a dynamic viscosity of between 50 and 2,000 mPa·s.

Advantageously, according to the invention, said kit further comprises at least one detergent component. Evidently, the kit according to the invention may have a sample comprising said at least one enzymatic component and said at least one detergent component formulated together or separately in solution or in solid form. When the sample is in solid form, a dilution/dissolution is carried out before the impregnation/saturation of the substrate.

Preferably, according to the invention, said sample of at least one detergent comprises at least one wetting agent and/or at least one sequestering agent and/or at least one dispersing agent.

In addition, in a particular embodiment, said one sample of at least one enzymatic component comprises at least one protease and/or one laccase and/or at least one polysaccharidase.

Other embodiments of a kit for the sampling of microorganisms present on a surface, in particular for the sampling of microorganisms having formed or not formed a biofilm on said surface, are indicated in the appended claims.

The present invention also relates to a use of an impregnated substrate according to the invention for the sampling of microorganisms present on a surface, in particular of microorganisms having formed or not formed a biofilm on said surface.

The present invention also relates to a use of a kit according to the invention for the sampling of microorganisms present on a surface, in particular of microorganisms having formed or not formed a biofilm on said surface.

Other forms of use of an impregnated substrate or a kit according to the invention for the sampling of microorganisms present on a surface, in particular for the sampling of microorganisms having formed or not formed a biofilm on said surface, are indicated in the appended claims.

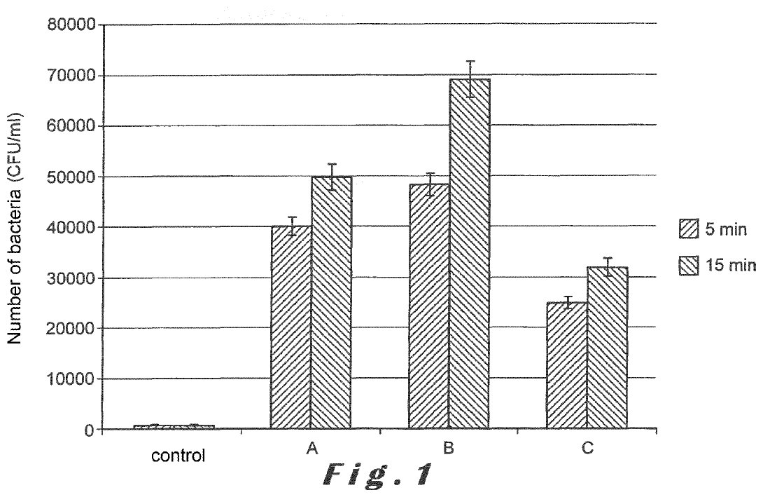

FIG. 1 shows the results (bacteria CFU/ml) obtained during the ATP-metry analyses of solutions obtained by removal from substrates impregnated with the compositions having different dynamic viscosities and having been applied for 5 or 15 min on a stainless-steel surface comprising the biofilm formed by Staphylococcus epidermidis and protein contamination (BSA).

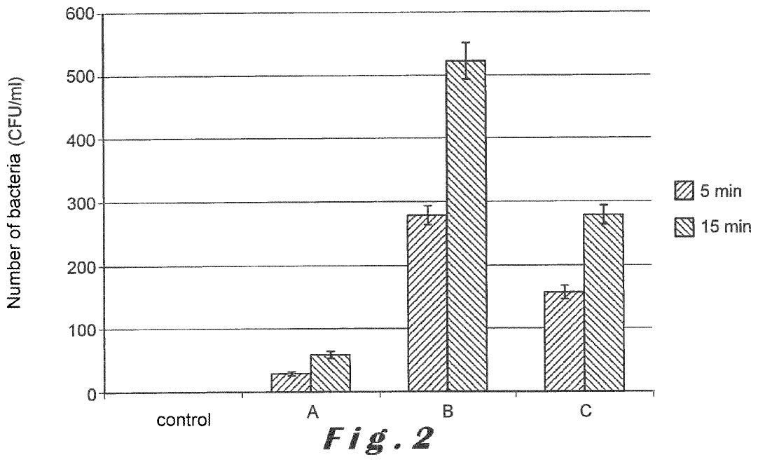

FIG. 2 shows the results (bacteria CFU/ml) obtained during the counting of the bacterial colonies developed by removal from substrates impregnated with the compositions having different dynamic viscosities and having been applied for 5 or 15 min on a stainless-steel surface comprising the biofilm formed by Staphylococcus epidermidis and protein contamination (BSA).

### EXAMPLES

### Example 1

### Effectiveness of Sampling Microorganisms Protected by a Biofilm from a Surface Depending on Whether the Substrate According to the Invention is Impregnated with a Composition whose Dynamic Viscosity is Different

Different dynamic viscosities of compositions impregnating a substrate according to the invention for the sampling of microorganisms present on a surface, in particular for the sampling of microorganisms having formed a biofilm on that surface, have been tested in terms of effectiveness of sampling the microorganisms from that surface. To do this, the following procedures were observed.
a) Procedure for the development of biofilm on stainless-steel surfaces
   Day −1: Inoculating a culture of Staphylococcus epidermidis in an Erlenmeyer flask containing 20 ml of sterile TSB medium (Tryptic Soy Broth) while maintaining stirring (120 rpm) at 35° C. for one night.
   Day 0:
      placing sterile stainless-steel surfaces in a sterile Petri dish with hydrophobic line drawn along the outline of the surfaces in order to allow the BSA stain to be kept on the surface (see below: procedure for depositing BSA stain);
      adding TSB medium to cover the stainless-steel surfaces;
      sampling 1 ml of the pre-culture of Staphylococcus epidermidis prepared the day before and placing this sampling in the Petri dish;
      stirring according to a rotating movement of the Petri dish, then incubating the latter for at least 20 h at 35° C.
   Day +1:
      removing the culture medium and rinsing the surfaces with 20 ml of sterile soft water twice;
      drying the surfaces at a temperature of 40° C. for at least one hour.
b) Procedure for depositing BSA stain (Bovin Serum Albumin)
   A layer of protein stain was added to the layer of biofilm formed according to the procedure of point a) above, this allowing the real situation where the biofilm may be very frequently combined with residual stains to be simulated. A solution containing 10 mg/ml of BSA was prepared before application of 7 ml of solution to each surface and a subsequent drying of the surfaces at a temperature of 40° C. maximum for several hours.
c) Procedure for sampling the contaminants present on the stainless-steel surfaces
   Sterile cloth substrates (Kaliwipe premium KW-P8030 from the supplier Conformat) were cut in order to be the same size as the surfaces to be analysed, i.e. 10×10 cm, and saturated with one of the following compositions heated to 45° C. beforehand:
      A) an aqueous solution containing: sodium laureth sulphate (detergent agent) in an amount of 5.4 v/v %, lauramine oxide (wetting agent) in an amount of 3 v/w/%, carboxymethyl inulin sodium salt (sequestering) in an amount of 0.11 v/v %, proteases in an amount of 0.31 v/v % (161 EX-type Savinase and 2.51 DX-type Alcalase), laccase in an amount of 0.005 v/v % (Novozym 51003), lipase in an amount of 0.07 v/v % (Lipolase 1001), alpha-amylase in an amount of 0.035 v/v % (Amplify 121), cellulase in an amount of 0.017 v/v % (Carezyme 45001) and mannanase in an amount of 0.005 v/v % (Mannaway 4/0L): dynamic viscosity of 8.4 mPa·s;
      B) an aqueous solution containing: sodium laureth sulphate (detergent agent) in an amount of 5.4 v/v %, sodium chloride (allowing the solution to thicken) in an amount of 1 v/v %, lauramine oxide (wetting agent) in an amount of 3 v/w/%, carboxymethyl inulin sodium salt (sequestering) in an amount of 0.11 v/v %, proteases in an amount of 0.31 v/v % (161 EX-type Savinase and 2.51 DX-type Alcalase), laccase in an amount of 0.005 v/v % (Novozym 51003), lipase in an amount of 0.07 v/v % (Lipolase 1001), alpha-amylase in an amount of 0.035 v/v % (Amplify 121), cellulase in an amount of 0.017 v/v % (Carezyme 45001) and mannanase in an amount of 0.005 v/v % (Mannaway 4/0L): dynamic viscosity of 396.5 mPa·s;
      C) an aqueous solution containing: sodium laureth sulphate (detergent agent) in an amount of 5.4 v/v %, sodium chloride (allowing the solution to thicken) in an amount of 1.2 v/v %, lauramine oxide (wetting agent) in an amount of 3 v/w/%, carboxymethyl inulin sodium salt (sequestering) in an amount of 0.11 v/v %, proteases in an amount of 0.31 v/v % (161 EX-type Savinase and 2.51 DX-type Alcalase), laccase in an amount of 0.005 v/v % (Novozym 51003), lipase in an amount of 0.07 v/v % (Lipolase 1001), alpha-amylase in an amount of 0.035 v/v % (Amplify 121), cellulase in an amount of 0.017 v/v % (Carezyme 45001) and mannanase in an amount of 0.005 v/v % (Mannaway 4/0L): dynamic viscosity of 2,213 mPa·s
         Each of these impregnated substrates was then placed on a vertical stainless-steel surface obtained as a result of steps a) and b) above. After the contact time of 5 or 15 min, the cloths were sterilely collected (retrieved). Then, for 30 seconds, a sterile, dry, non-saturated cloth was applied to the surfaces in order to absorb the sampling solution still present there. For a given stainless-steel surface, the saturated and non-saturated cloths were then gathered into a sterile packet.
d) Procedure for removing collected samples
   In order to remove, from the cloths resulting from the procedure under point c) above, the molecules (contaminants) collected from the stainless-steel surfaces, 90 ml of Thio solution was added to the sterile packet before its passage through the mixer for 2 minutes in order to obtain a solution ready to be analysed. Thio solution is a sterile solution containing several components having the ability to neutralise the biocide effect of the products which could be used on the surfaces (disinfectants used during the cleaning/disinfection phase in the industry, for example). This solution comprises 3 g/l polysorbate 80, 0.3 g/l lecithin, 0.1 g/l L-histidine, 0.5 g/l sulphate thiosulphate. These components are diluted in the demineralised water and the volume is brought to 11. The solution is sterilised in an autoclave before use.
e) Analyses of the solutions
   Two types of analyses were carried out:
   1. Second generation ATP-metry (ATP-2G)
      The analysis of the solutions obtained after removal was carried out according to the ATP-2G technique which is a technology allowing the quantity of intracellular ATP in a sample to be measured in a few minutes. This dosage quantifies all the microorganisms living in the liquid medium. This measurement was carried out with the QGA 2G kit marketed by the Aqua-tools® company by observing the instructions and procedure of the manufacturer which indicates how to convert the ATP measurement into the number of colonies.
      The results obtained are given in FIG. 1. As can be seen, the ATP-metric analysis shows a greater level of contamination in the solution having a dynamic viscosity of between 50 and 2,000 mPa·s than for the solutions having lower or higher viscosities. This means that, according to a dynamic viscosity of between 50 and 2,000 mPa·s of the composition impregnating the sampling substrate according to the invention, a sampling of all the bacterial contaminants and similar is carried out. At least, an improved sampling is carried out with a sampling substrate impregnated with a composition having such a dynamic viscosity according to the invention.
2. Bacterial counting
   The solutions obtained from the procedure of point d) above were also spread in Petri dishes containing the non-specific, universal PCA medium (n=3), the dishes then being incubated at 35° C. for 24 h before counting the colonies which developed there.
      The results obtained are given in FIG. 2. As can be seen, the same trend as observed for the ATP-metry occurs, that is that a greater contamination of the solutions is seen in the solutions having a dynamic viscosity of between 50 and 2,000 mPa·s. The levels of contamination detected were much weaker than for the ATP-metry, this being explained by the fact that the microorganisms of the biofilm are poorly cultivated in Petri dishes (stressed condition which makes them undetectable by this common microbiological technique).
      This again means that with such a dynamic viscosity of the composition impregnating the sampling substrate according to the invention, a sampling of all the bacterial contaminants and similar is carried out. At least, an improved sampling is carried out with a sampling substrate impregnated with a composition having such a dynamic viscosity according to the invention.

### Example 2

### Adherence of a Sampling Substrate According to the Invention onto Vertical and Horizontal Surfaces according to the Dynamic Viscosity of the Impregnated Composition in this Substrate

The sampling procedure as described in example 1 is applied with the three solutions (A, B, C) of different viscosities. For each solution (composition), a test is carried out on a vertical surface and on a horizontal surface as being able to collect under a surface may turn out to be particularly interesting in the industry.

Several parameters were identified following the application of the sampling substrates:
saturation of the cloth with the composition and distribution of these;
holding the cloths on the surface;
runoff of the sampling composition from the cloth onto the surface; and
distribution of the sampling composition on the surface when the cloth is removed from the surface: the surface should preferably be entirely covered with a fine layer of the sampling composition when the first cloth is removed.

Table 1 below gives the results obtained for these different parameters.

TABLE 1

| Solution | On a vertical surface | On a horizontal surface |
|---|---|---|
| A: | good distribution of the | good distribution of the |
| viscosity of | sampling composition on | sampling composition on |
| 8.4 mPa · s | the cloth and on the | the cloth and on the |
| | surface. | surface |
| | the cloth remains adhered | the cloth remains adhered |
| | for 15 min but runoff of | for 15 min, no runoff |
| | the composition after 1 min | |
| | for a length greater | |
| | than 22 cm. | |
| B: | good distribution of the | good distribution of the |
| viscosity of | sampling composition on | sampling composition on |
| 396.5 mPa · s | the cloth and on the | the cloth and on the |
| | surface. | surface. |
| | the cloth remains adhered | the cloth remains adhered |
| | for 15 min, no runoff, and | for 15 min, no runoff, and |
| | remains well adhered to | remains well adhered to |
| | the surface. | the surface. |
| | after removal of the | after removal of the |
| | cloth, 100% of the surface | cloth, 100% of the surface |
| | is covered with | is covered with |
| | composition. | composition. |
| C: | bad distribution of the | bad distribution of the |
| viscosity of | sampling composition on | sampling composition on |
| 2,213 mPa · s | the cloth and on the | the cloth and on the |
| | surface, a portion of the | surface, a portion of the |
| | cloth remains dry. | cloth remains dry. |
| | after removal of the | after removal of the |
| | cloth, a portion of the | cloth, a portion of the |
| | surface is not covered | surface is not covered |
| | (90% of surface covered). | (90% of surface covered). |
| | the cloth remains adhered | the cloth unsticks at the |
| | for 15 min, no runoff. | corners. |

A comparison of the different parameters identified allows it to be noted that it is the sampling substrates according to the invention impregnated with a composition having a dynamic viscosity of between 50 and 2,000 mPa·s which has the best characteristics allowing it to be guaranteed that the sampling will be appropriate and representative of the populations of microorganisms present on the surfaces.

It is understood that the present invention is in no way limited to the embodiments described above and that modifications may be applied without departing from the scope of the appended claims.

1. Substrate impregnated with at least one composition comprising at least one enzymatic component for the sampling of microorganisms present on a surface, in particular for the sampling of microorganisms having formed or not formed a biofilm on said surface, characterised in that said at least one composition has a dynamic viscosity of between 50 and 2,000 mPa·s.

2. Substrate according to claim 1, characterised in that said at least one enzymatic component comprises at least one protease and/or at least one laccase and/or at least one polysaccharidase.

3. Substrate according to claim 1, characterised in that said substrate is a wipe or a sponge or a cloth or any other type of appropriate substrate.

4. Substrate according to claim 1, characterised in that said at least one composition comprising at least one enzymatic component has a pH of between 5 and 11.

5. Substrate according to claim 1, characterised in that said substrate and said at last one composition comprising at least one enzymatic component are sterile.

6. Substrate according to claim 1, characterised in that said at least one composition comprising at least one enzymatic component further comprises at least one detergent component.

7. Substrate according to claim 6, characterised in that said at least one detergent component comprises at least one wetting agent and/or at least one sequestering agent and/or at least one dispersing agent.

8. Substrate according to claim 6, characterised in that said at least one detergent component comprises a proportion of sequestering agent of between 1 and 10 wt %, a proportion of dispersing agent of between 1 and 10 wt % and a proportion of wetting agent of between 1 and 30 wt % with respect to the total weight of the detergent component.

9. Method for the sampling of microorganisms present on a surface, in particular sampling microorganisms having formed or not formed a biofilm on said surface, using a substrate according to claim 1, said method comprising the following steps:
impregnating a substrate with a predetermined quantity of at least one composition comprising at least one enzymatic component and having a dynamic viscosity of between 50 and 2,000 mPa·s;
applying said impregnated substrate to said surface over a predetermined period of time in order to collect, from said surface, microorganisms having formed or not formed a biofilm thereupon; and
removing said substrate from said surface.

10. Method according to claim 9, characterised in that said predetermined period of application time of said impregnated substrate to said surface is between 1 and 45 minutes and is preferably 15 minutes.

11. Method according to claim 9, characterised in that it comprises a prior step of heating said at least one composition comprising said at least one enzymatic component to a temperature of between 15 and 65° C., preferably to a temperature equal to 45° C.

12. Method according to claim 9, characterised in that it comprises an additional step of swabbing said surface so the microorganisms not collected by said impregnated substrate are collected by swabs.

13. Method according to claim 9, characterised in that it comprises an additional step of removing said collected microorganisms in a sterile removal solution.

14. Method according to claim 13, characterised in that it comprises an additional step of quantification and/or identification of said removed microorganisms.

15. Kit for the sampling of microorganisms present on a surface, in particular for the sampling of microorganisms having formed or not formed a biofilm on said surface, said kit comprising:
a substrate, for example a fabric-type substrate; and
a sample of at least one enzymatic component in solution or in solid form.

16. Kit according to claim 15, characterised in that it further comprises a sample of at least one detergent component.

17. Kit according to claim 16, characterised in that said sample of at least one detergent comprises at least one wetting agent and/or at least sequestering agent and/or at least one dispersing agent.

18. Kit according to claim 9, characterised in that said sample of at least one enzymatic component comprises at least one protease and/or at least one laccase and/or at least one polysaccharidase.

19. Use of a substrate according to claim 1 for the sampling of microorganisms present on a surface, in particular for the sampling of microorganisms having formed or not formed a biofilm on said surface.

20. Use of a kit according to claim 15 for the sampling of microorganisms present on a surface, in particular for the sampling of microorganisms having formed or not formed a biofilm on said surface.
